(19) United States
(12) Patent Application Publication
(10) Pub. No.: US 2006/0153731 A1
(43) Pub. Date: Jul. 13, 2006

(54) Apparatus for volatile liquid dissemination

(75) Inventors: Colin Brown, Bracknell (GB); Guy Edward Naish, Bicester (GB); Kishen Gohil, New Malden (GB)

Correspondence Address: Norris, McLaughlin & Marcus P.A., 18th Floor, 875 Third Avenue, New York, NY, 10022, US

(73) Assignee: Givaudan SA, Vernier (CH)

(21) Appl. No.: 11/328,004

(22) Filed: Jan. 9, 2006

(30) Foreign Application Priority Data
Jan. 12, 2005 (GB) 0500480.9

(51) Int. Cl. A61L 9/00

(52) U.S. Cl. 422/5; 422/123

(57) ABSTRACT

An apparatus adapted to provide to at least one atmosphere a volatile liquid whose presence therein in vapour form is desired, the apparatus comprising a source of volatile liquid, means of emitting this liquid to the atmosphere in a vapour phase and control means adapted to regulate the quantity of the emission, control being exerted remotely by means of an electromagnetic signal. The apparatus preferably comprises part of a system of apparatus for releasing liquid into a plurality of atmospheres, all of which are centrally controlled by radio frequency signals. The apparatus permits the central regulation of the emission of fragrance in a number of different atmospheres, for example, a plurality of rooms in a house.

This invention relates to apparatus for dissemination of volatile substances into an atmosphere and particularly to such apparatus that can be remotely activated.

Apparatus for dissemination of a volatile substance, such as a fragrance, an odour masking agent or an insecticide into an atmosphere are well known and widely used. These apparatus are generally simple and therefore cheap to manufacture, and for basic uses they are completely satisfactory. However, if more sophistication is desired, such as multi-room installations with different requirements in every room, and even the use of different apparatus in different rooms at different times, the simple devices known to the art are simply incapable of delivering the desired levels of performance.

It has now been found that it is possible to provide such apparatus, which satisfy the requirements of the most demanding multi-room application. The invention therefore provides an apparatus adapted to provide to at least one atmosphere a volatile liquid whose presence therein in vapour form is desired, the apparatus comprising a source of volatile liquid, means of emitting this liquid to the atmosphere in a vapour phase and control means adapted to regulate the quantity of the emission, control being exerted remotely by means of an electromagnetic signal.

The basic apparatus has a source of volatile liquid and means for emitting this liquid into an atmosphere. The source is typically an enclosed reservoir, preferably one adapted to easy refilling or replenishment, for example, by providing the reservoir in easily detachable form, so that a full reservoir can be quickly inserted in place of an empty one. This also allows the possibility of a quick and easy change of the nature of the liquid. This is especially useful when the volatile liquid is a fragrance, as the nature of the atmosphere desired can be easily changed.

The means for emitting liquid as a vapour into an atmosphere may be chosen from any of the large number that is known to the art. A common example is an evaporative surface, typically a porous wick that has one end immersed in the liquid and the other end exposed to the atmosphere in which the presence of the liquid is desired. Such wicks can take many sizes and shapes and be made from a variety of materials. Dissemination into the atmosphere may be purely evaporative, or it may be assisted by one or more auxiliary means, for example, a heating element or an electric fan.

The apparatus additionally comprises control means adapted to regulate the quantity of the emission. Quantity can be regulated by one or both of (a) regulation of the weight emitted per unit time, and (b) the duration of the emission. There are many ways of regulating (a), for example, by altering the temperature of a heating element, the air velocity produced by a fan and the size of apertures of a casing separating the volatile liquid emitter from the atmosphere. Similarly, (b) can be regulated by varying the time of operation of a fan or a heating element. The skilled person will realise that there are many other embodiments not mentioned here that lie within the skill of the art and that fall within the scope of the invention. A combination of two or more such elements is possible and permissible.

The electrical power necessary to achieve the control hereinabove described may be provided by any convenient means, for example, from batteries, solar cells or directly from the mains electrical supply, either via an electrical lead or by being directly plugged into a power point.

An important feature of the apparatus of this invention is the regulation of the control means by electromagnetic signal. The frequencies of the electromagnetic spectrum range from very high (gamma and cosmic radiation) to very low (long wave radio signals), and any of these wavelengths that are suitable for control purposes may be used. For example, the signal may be in the infra-red, as used in remote control apparatus for televisions and stereo equipment. While these are suitable for use in this invention, they are hampered by being “line of sight” devices that can only operate when the remote control device is pointed at them or at least in their general direction. In a preferred embodiment, the electromagnetic signal is a radio frequency signal. Such frequencies do not necessitate line of sight or pointing. It need not even be in the same room. Any radio frequency that is permitted by relevant authorising bodies may be used. Typically suitable frequencies are short-range frequencies in the 100-500 MHz range, used typically for remote outside sensors for domestic thermometers.

A further advantage of radio control is that a multiplicity of apparatus may be used and operated from a single control means. In its simplest form, all apparatus respond to a common radio frequency and may be activated simultaneously. Alternatively, a master apparatus may be activated by, for example, an infrared-type remote controller, and in addition to that master apparatus emitting liquid, it could signal to other apparatus by radio frequency signal that they should also start emitting. The invention therefore provides a system adapted to provide to a multiplicity of atmospheres at least one volatile liquid whose presence therein in vapour form is desired, comprising a plurality of liquid-providing apparatus as hereinabove described, at least one per atmosphere, all apparatus being controllable by radio frequency signals.

In a particularly useful variation of this embodiment, all apparatus are equipped with dual master-slave capability, so that any apparatus can act as the master and signal to the others, which then become the slaves. This permits the commanding of one apparatus to perform a particular function, and all the others simultaneously perform the same function. Thus, for example, all apparatus may be equipped with a boost button, to boost the dissemination of volatile liquid into the air, typically by increasing the speed of a fan. On this being pressed on any apparatus, there is the possibility of ensuring that this apparatus signals this to the other apparatus, which then boost their outputs, as if the individual buttons on those apparatus were pressed. This allows increased dissemination without the need to carry a remote control apparatus. Alternatively, the boost button may be on a remote controller, and pressing the button has the effect of activating all apparatus.

It is also possible to have a plurality of apparatus working in different places in different manners. For example, in the case of fragrance emission, there may be apparatus in a number of different rooms of a house or a hotel, and the emission of fragrance in each room may be individually regulated, simply by giving each apparatus it own radio frequency, to which only it responds. Thus it would be possible to regulate the performances of a plurality of individual apparatus, in terms of different emission times and different emission quantities. In a further embodiment, a number of individual apparatus, each having a different volatile liquid (typically a number of different fragrances) may be combined in a single unit and the nature of the fragrance, as well as the emission time and volume, may also be regulated.

The invention therefore includes means adapted to provide at least one volatile liquid into a plurality of atmospheres, there being present in each atmosphere at least one apparatus as hereinabove described, each apparatus being responsive to a specific radio signal such that the emission of liquid into each atmosphere may be individually and remotely controlled from a central control apparatus.

The invention further provides a method of providing a volatile liquid in each of a plurality of atmospheres, comprising the provision of at least one apparatus as hereinabove described in each atmosphere and controlling the quantity of emission by means of a radio signal from a remote central control apparatus.

In a further embodiment, individual apparatus may be equipped with microprocessor controls and memory, such that the individual apparatus can be programmed in advance, and there is no need to reset all the apparatus on each occasion.

The invention permits a versatility of use that is unknown in the art. A number of different liquids can be released in different atmospheres (for example, different rooms) at different times and in different quantities. The invention therefore also provides a method of providing volatile liquid in a plurality of atmospheres, comprising providing means for disseminating liquid in each atmosphere in a desired quantity, the means in each atmosphere being independently controlled by means of a radio frequency signal.

The apparatus and its ancillary equipment are easily and cheaply made from known components and work reliably.

The invention is further described with reference to the drawings, which depict preferred embodiments and which do not in any way limit the scope of the invention.

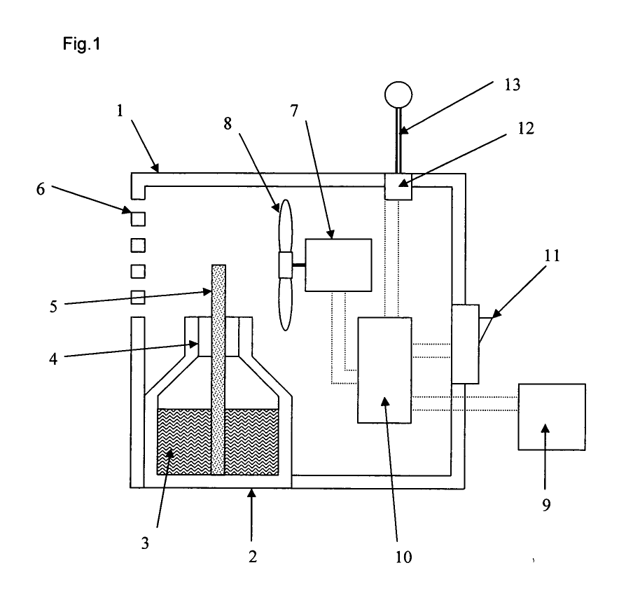

FIG. 1 is a schematic cross-section of a master fragrance-emitting unit.

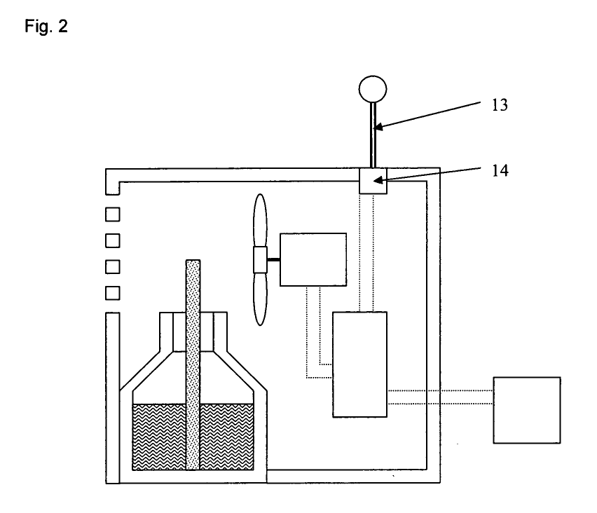

FIG. 2 is a schematic cross-section of a slave fragrance-emitting unit.

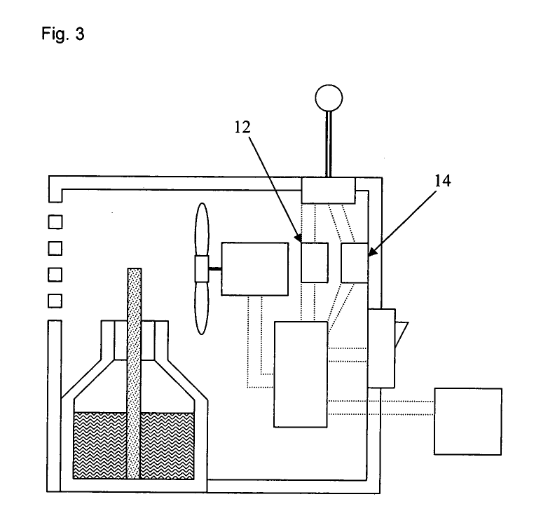

FIG. 3 is a schematic cross-section of a fragrance-emitting unit that can act as both master and slave.

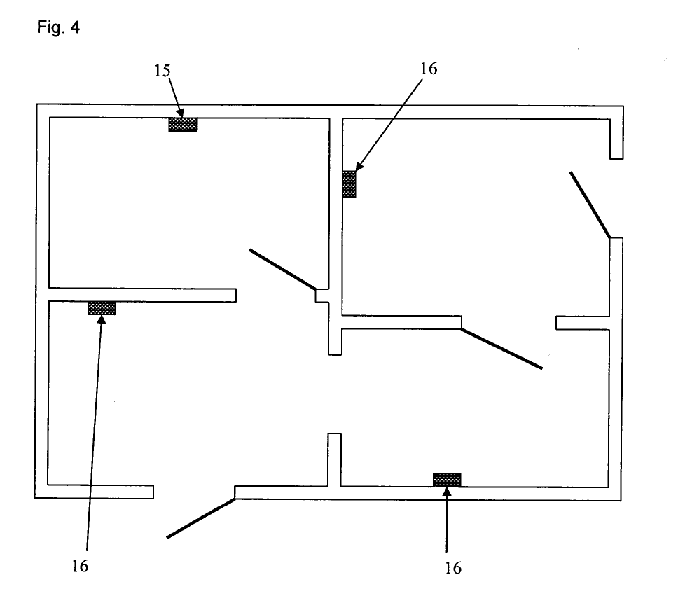

FIG. 4 is a schematic depiction of the location of units in a household situation.

The master unit of FIG. 1 comprises a main body member 1 enclosing a replaceable reservoir 2 containing volatile liquid 3 which is drawn out of the reservoir 2 via a porous wick 4 that is held in the reservoir 2 by a plastic insert 5. Components 2-5 together form a unit that can easily be removed and replaced. The wick 4 is positioned behind a vent or grill 6 in the body member 1. An impeller 8 driven by an electric motor 7 enhances the volatilisation of the liquid into the atmosphere. The unit may comprise a heating element in addition to or instead of the impeller, to enhance volatilisation.

The unit has a power source 9 which may be battery, solar or mains power. The power is controlled by a control circuit 10 which regulates power to the other elements in the device. This circuit may optionally have a timer to provide pulses of power to the motor 7 or it may have a voltage regulator to provide increased/decreased power to the motor or heater on command, thus altering the output of volatile material from the device. The unit has a switch or series of controls 11 by which an operator can activate the functions of the unit. There is also a radio frequency transmitter 12 and antenna 13. In use, on activation of power, the unit runs to a set sequence but activation of the appropriate control 11 performs a pre-set change in the function, such as a boost in fan speed and also transmits an RF signal to other units within range to effect the same change.

The slave unit of FIG. 2 has essentially the same construction as the master unit, but it lacks the control 11 of that unit and the antenna 13 is connected to an RF receiver 14. The control circuit in this case is designed to recognise the incoming RF signal and effect a pre-set change in the unit's function.

The apparatus of FIG. 3 has obvious similarities with those of FIGS. 1 and 2, but in this case, it has both a transmitter 12 and receiver 14, enabling it to act as both master and slave. The actuation of this apparatus will cause it to send a signal to other apparatus, commanding them to perform the same actuation.

FIG. 4 shows a housing layout with four rooms, one room containing a master unit 15 and the other three rooms containing slave units 16. Although none of the slave units are in line of sight of the master unit, they are all within range of the master transmitter and thus will respond to any transmitted commands.

1. An apparatus adapted to provide to at least one atmosphere a volatile liquid whose presence therein in vapour form is desired, the apparatus comprising a source of volatile liquid, means of emitting this liquid to the atmosphere in a vapour phase and control means adapted to regulate the quantity of the emission, control being exerted remotely by means of an electromagnetic signal.

2. An apparatus according to claim 1, wherein the electromagnetic signal is a radio frequency signal.

3. An apparatus according to claim 2, in which the signal frequency is in the range 100-500 MHz.

4. A system adapted to provide to a multiplicity of atmospheres at least one volatile liquid whose presence therein in vapour form is desired, comprising a plurality of liquid-providing apparatus according to claim 1, at least one per atmosphere, all apparatus being controllable by radio frequency signals.

5. A system according to claim 4, wherein all apparatus respond to a common radio frequency and are capable of being activated simultaneously.

6. A system according to claim 4, wherein the system comprises a master apparatus that can signal by means of radio frequency signal to other slave apparatus that it or they should commence emitting liquid.

7. A system according to claim 6, wherein all apparatus have dual master-slave capability, allowing any one apparatus to act as master and command the other apparatus.

8. A system according to claim 4, wherein each apparatus responds to a radio frequency individual to it.

9. Means adapted to provide at least one volatile liquid into a plurality of atmospheres, there being present in each atmosphere at least one apparatus as hereinabove described, each apparatus being responsive to a specific radio signal such that the emission of liquid into each atmosphere may be individually and remotely controlled from a central control apparatus.

10. A method of providing volatile liquid in a plurality of atmospheres, comprising providing means for disseminating liquid in each atmosphere in a desired quantity, the means in each atmosphere being independently controlled by means of a radio frequency signal.
